(19) United States
(12) Patent Application Publication
(10) Pub. No.: US 2020/0338259 A1
(43) Pub. Date: Oct. 29, 2020

(54) PACKAGING FOR A MEDICAL DEVICE

(71) Applicant: Becton, Dickinson and Company, Franklin Lakes, NJ (US)

(72) Inventors: Emilie Mainz, Baltimore, MD (US); Alex Chaves, Tyngsboro, MA (US)

(73) Assignee: Becton, Dickinson and Company, Franklin Lakes, NJ (US)

(21) Appl. No.: 16/768,620

(22) PCT Filed: Nov. 30, 2018
(86) PCT No.: PCT/US2018/063352

§ 371 Date: May 29, 2020

Related U.S. Application Data
(60) Provisional application No. 62/607,728, filed on Dec. 19, 2017.

(51) Int. Cl. A61M 5/00; A61M 5/158; B65B 7/28

(52) U.S. Cl. CPC A61M 5/002; A61M 5/158; A61M 2005/1585; A61M 2205/6063; A61M 2205/6072; B65B 7/2864

(57) ABSTRACT

A method of packaging a medical device, such as an infusion set (10), comprises forming a base (28) having a cavity (29), placing the medical device (10) within the cavity (29) of the base (28), applying an instruction guide (24) to an inner surface (22) of a peelable cover (20), applying adhesive (26) between the base (28) and the inner surface (22) of the peelable cover (20) surrounding the instruction guide (24), and sealing the peelable cover (20) to the base (28) via the adhesive (26).

### CROSS-REFERENCE TO RELATED APPLICATION

This application claims the benefit under 35 U.S.C. § 119(e) of Provisional Application No. 62/607,728, filed on Dec. 19, 2017, which is incorporated herein by reference in its entirety.

### FIELD OF THE INVENTION

The present invention generally relates to medical devices and to methods for making and packaging such devices.

### BACKGROUND OF THE INVENTION

A large number of people with diabetes use some form of daily insulin therapy to maintain close control of their glucose levels. Currently, there are two principal modes of daily insulin therapy. The first mode includes syringes and insulin pens. These devices are simple to use and are relatively low in cost, but they require a needle stick at each injection, typically three to four times per day. The second mode includes infusion pump therapy, which entails the use of an insulin pump. Although the initial cost of the pump can be significant, the overwhelming majority of patients who have used pumps prefer to remain with pumps. This is because infusion pumps, although more complex than syringes and pens, offer the advantages of continuous infusion of insulin, precision dosing, and programmable delivery schedules. This results in closer blood glucose control, which can result in improved health outcomes for the patients.

The use of an infusion pump requires the use of a disposable component, typically referred to as an infusion set, line set, extension set or pump set, which conveys the insulin from a reservoir within the pump into the skin of the user. An infusion set typically consists of a pump connector, a length of tubing, and a hub or base from which an infusion cannula (i.e., an infusion needle or a flexible catheter) extends. The hub or base has an adhesive, which retains the base on the skin surface during use, which may be applied to the skin manually or with the aid of a manual or automatic insertion device.

Infusion sets are typically individually packaged in sterile packaging and then placed in cartons having multiple infusion sets. Generally, there is one instruction guide or “quick start guide,” in loose paper form, that is packaged inside the carton and provides instructions on how to use the infusion sets. Problems can arise during use of the infusion sets when the quick start guide is misplaced. Also, it is inconvenient to have to safely store the quick start guide for reference when each infusion set is used.

### SUMMARY OF THE INVENTION

It is an aspect of the present invention to provide an infusion set or other medical device individually packaged with an instruction guide. Specifically, a peelable cover is removed from a base to access the infusion set for use. The instruction guide is advantageously disposed on the inner surface of the peelable cover. Typically, in the prior art, the inner surface of the peelable cover is blank. Accordingly, each infusion set conveniently includes the instruction guide to provide a quick reference to the user without the risk of being misplaced. Further, infusion set installation and operational compliance is improved. Also, the peelable cover including the instruction guide advantageously reduces waste by eliminating the need for a separate carton insert as in the prior art.

The foregoing and/or other aspects of the present invention can be achieved by providing a method of packaging a medical device, the method comprising forming a base having a cavity, placing the medical device within the cavity of the base, applying an instruction guide to an inner surface of a peelable cover, applying adhesive between the base and the inner surface of the peelable cover surrounding the instruction guide, and sealing the peelable cover to the base via the adhesive.

The foregoing and/or other aspects of the present invention can also be achieved by providing a method of using a medical device with an instruction guide, the method comprising peeling a peelable cover adhered to a base which encloses the medical device, removing the medical device from the base, viewing the instruction guide on an inner surface of the peelable cover after the peeling and using the infusion set based on information in the instruction guide.

Additional and/or other aspects and advantages of the present invention will be set forth in the description that follows, or will be apparent from the description, or may be learned by practice of the invention.

### BRIEF DESCRIPTION OF THE DRAWINGS

The above aspects and features of the present invention will be more apparent from the description of the exemplary embodiments of the present invention taken with reference to the accompanying drawings, in which:

FIG. 1 illustrates a top perspective view of a first exemplary embodiment of various components of an infusion set;

FIG. 2 illustrates a top perspective view of the infusion set of FIG. 1 enclosed in a transparent base;

FIG. 3 illustrates a bottom view of the infusion set of FIG. 2 enclosed in the base and a peelable cover;

FIG. 4 illustrates a bottom perspective view of the infusion set of FIG. 2 enclosed in the base that is partially opened;

FIG. 5 illustrates a bottom perspective view of the infusion set of FIG. 2 enclosed in the base that is more fully opened;

FIG. 6 illustrates a bottom perspective view of the infusion set of FIG. 2 enclosed in the base that is fully opened;

FIG. 7 illustrates a side view of the infusion set of FIG. 1 adhered to the skin of a patient; and

FIG. 8 illustrates a perspective view of an inserter and a pump each configured to engage the infusion set of FIG. 1.

### DETAILED DESCRIPTION OF EXEMPLARY EMBODIMENTS

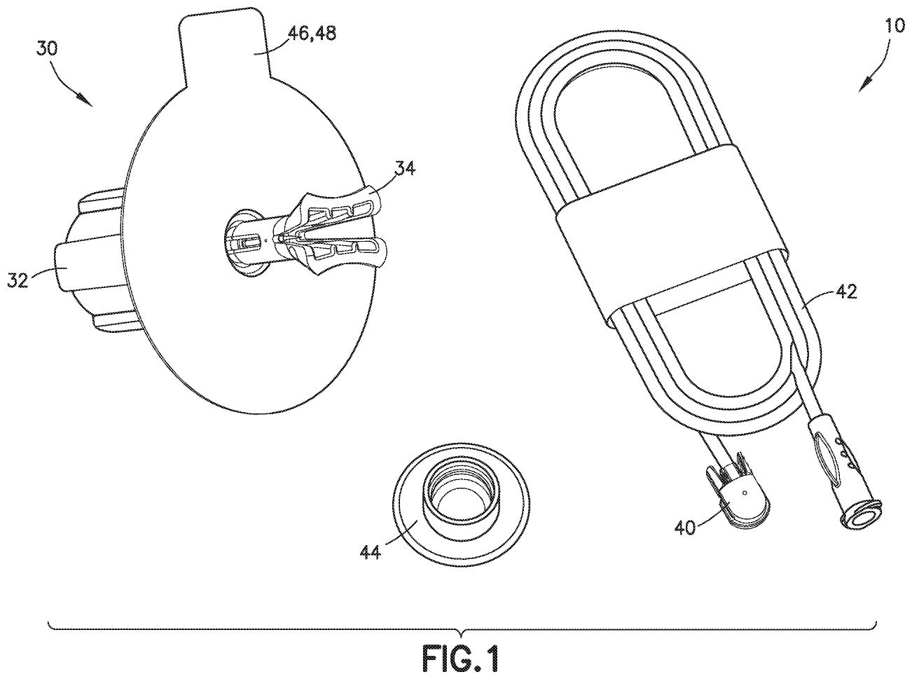

FIG. 1 illustrates an infusion set 10 including an insertion set 30. The insertion set 30 includes a needle housing 32 and a needle guard 34 enclosing a cannula (not shown). The needle housing 32 encloses a needle (not shown) used to insert the cannula into a skin of a patient. The needle housing 32 and the needle guard 34 are both used to protect an infusion set huh (not shown), the needle and cannula prior to use.

To begin operation of the infusion set 10, the needle housing 32 and the needle guard 34 are removed. Next, the needle and cannula are inserted into the skin of the patient either manually or with an inserter, as further described below. After the cannula is placed in the patient, the needle is removed from the infusion set 10.

The infusion set 10 further includes an adhesive patch 46 having an adhesive 48. The adhesive patch 46 is fixed to the cannula of the insertion set 30 and is configured to secure the insertion set 30 to the skin of the patient via the adhesive 48. The adhesive patch 46 surrounds the insertion set 30.

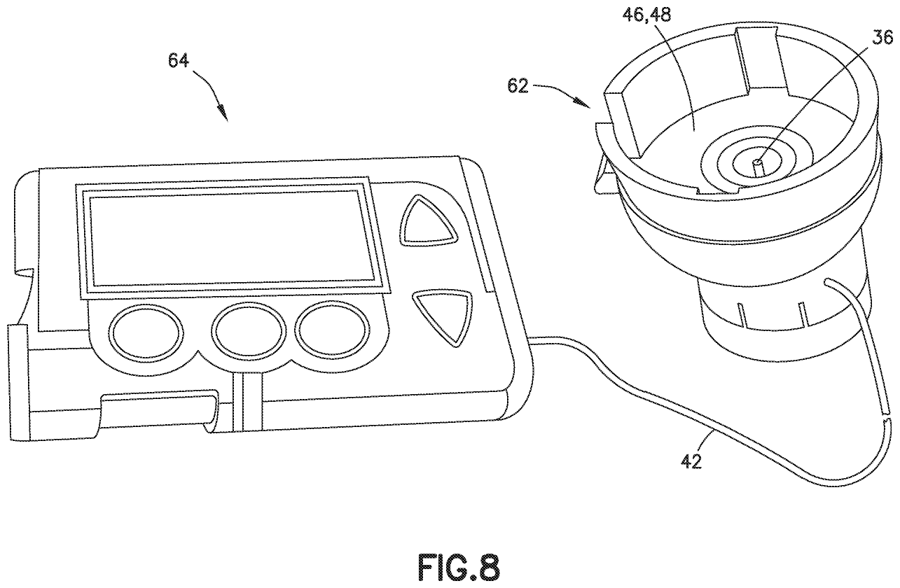

The infusion set 10 also includes tubing 42 used to connect the infusion set 10 to a pump 64 as illustrated in FIG. 8. The tubing 42 connects to the infusion set hub 50 of the infusion set 10 via a fluid connector 40, When the fluid connector 40 is disengaged from the infusion set 10, a disconnect cover 44 is placed on the infusion set 10 to prevent leakage.

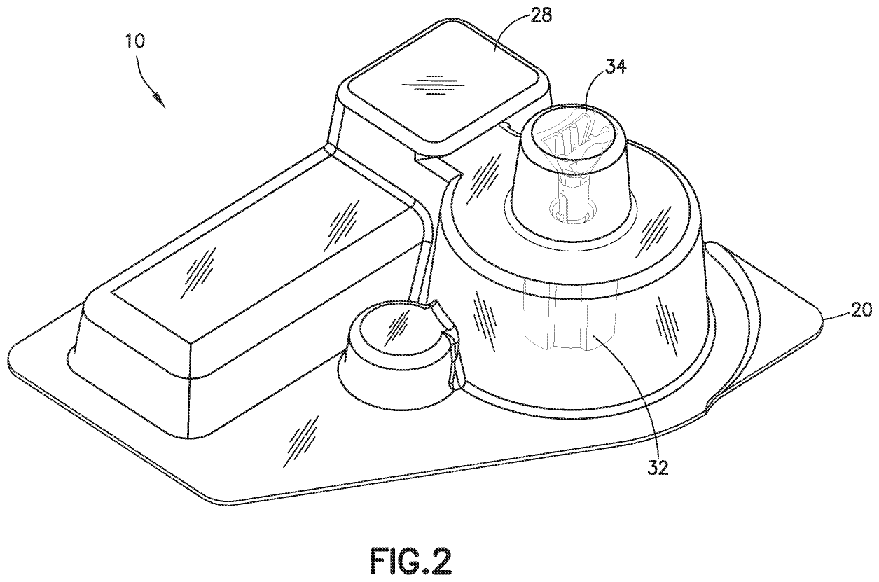

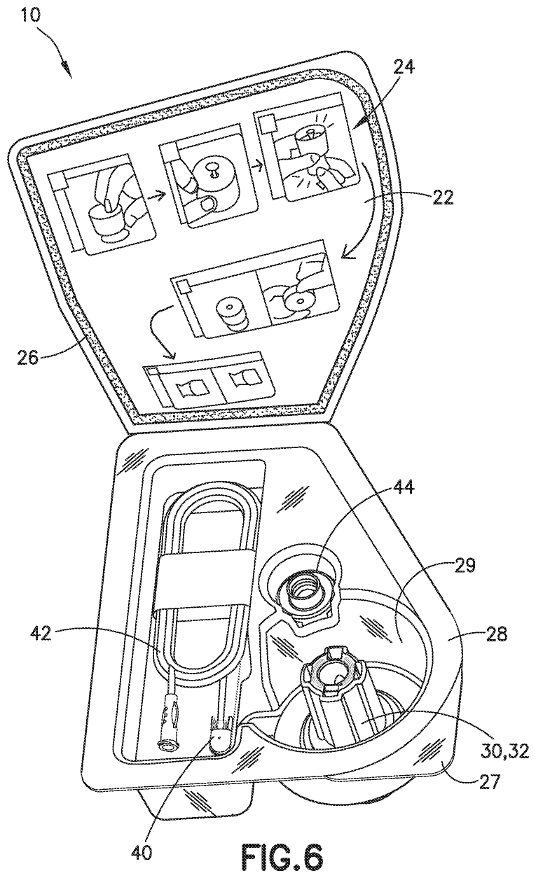

FIG. 2 illustrates the components of the infusion set 10 described above being packaged in a base 28 and sealed by a peelable cover 20. The base 28 is preferably formed to include a plurality of cavities 29, as illustrated in FIG. 6, to support the components of the infusion set 10. The base 2$ is advantageously transparent and clear (untinted) to allow the user to see the contents of the infusion set 10 inside, before opening the package. In an alternative embodiment, the base 28 can be tinted, translucent or opaque.

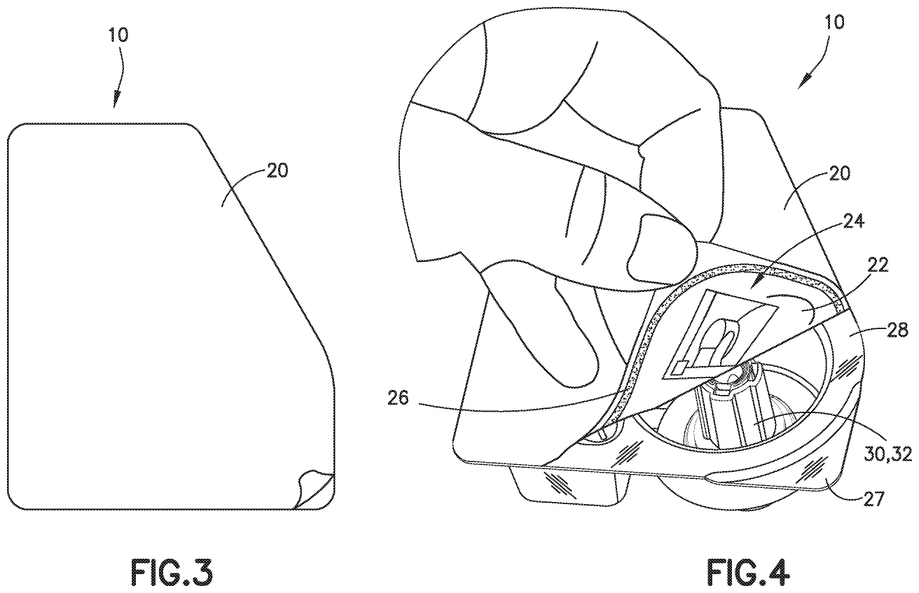

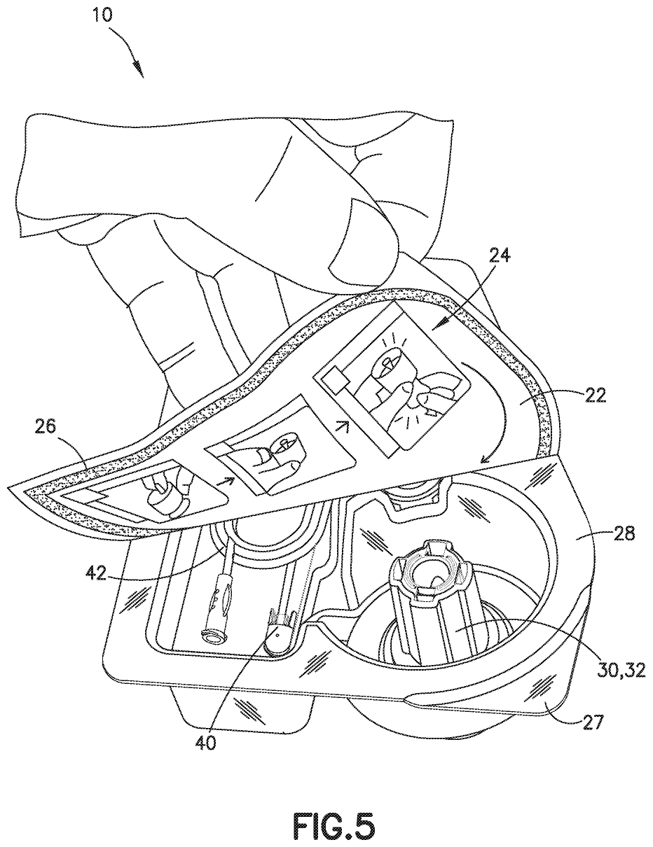

The peelable cover 20, as illustrated in FIG. 3, has a flat surface and is adhered to the base 28 via an adhesive 26. Preferably, the peelable cover 20 is made of Tyvek®, although other similar materials can be used. FIG. 3 illustrates the peelable cover 20 sealed to the base 28 and fully enclosing the components of the infusion set 10. FIGS. 4-6 illustrate the peelable cover 20 being gradually pulled from the base 28 to gain access to the components of the infusion set 10.

FIGS. 4-6 illustrate that the peelable cover 20 advantageously includes a “quick start” guide, also known as instructions for use (IFU) or an instruction guide 24. The instruction guide 24 is applied to the inner surface 22 of the peelable cover 20 either integrally (as by priming) or separately (as a label). Preferably, the instruction guide 24 is integrated on an inner surface 22 of the peelable cover 20. That is, the instruction guide 24 is not physically separate from the peelable cover 20, In an alternate embodiment, the instruction guide 24 is a separate label that is adhered to the inner surface 22 of the peelable cover 20.

As illustrated in FIG. 3, the instruction guide 24 is enclosed and sealed within the peelable cover 20 and the base 28, If the base 28 is clear and transparent, the instruction guide 24 can be seen, at least partially, through the base 28 without removing the peelable cover 20 from the base 28. This is advantageous in that it allows the user to see that the package includes an instruction guide before the package is opened.

Alternately, the base 28 can be opaque to provide an orderly and sleek appearance. Although the infusion set 10 and instruction guide 24 cannot be seen prior to opening, both the infusion set 10 and the instruction guide 24 would gradually be displayed during opening.

The instruction guide 24 comprises at least one of a printed instruction or a set of printed instructions, a written instruction or a set of written instructions, an image, a scannable barcode, and a scannable QR code. The instruction guide 24 provides information comprising of at least one of a product specification, assembly instructions, installation instructions, and operating instructions. Any combination of text or symbols providing information related to the infusion set 10 can be used in the instruction guide 24.

Applying the instruction guide 24 to an inner surface 22 of the peelable cover 20 advantageously provides an instruction guide 24 for each infusion set 10. This frees the user from having to carry a separate instruction guide also minimizes waste. The instruction guide 24 applied to the peelable cover 20 advantageously makes the instruction guide 24 difficult to misplace and provides a quick reference. Improved user compliance with the information in the instruction guide 24 is also advantageously achieved.

The peelable cover 20 and/or the base 28 includes adhesive 26 that allows the peelable cover 20 to engage and be sealed to the base 28. The adhesive 26 is preferably applied to an outer perimeter of the instruction guide 24 and on the inner surface 22 of the peelable cover 20 but the adhesive 26 can also be applied on an outer perimeter of the base 28. The adhesive 26 is preferably heat sealed to fix and provide a complete sealing interface between the peelable cover 20 to the base 28. Accordingly, the instruction guide 24 is sealed between the peelable cover 20 and the base 28 while not being obstructed.

The base 28 includes a pull tab 27 that is not sealed to the peelable cover 20. A small outer edge area of the peelable cover 20 opposing the pull tab 27 of the base 28 has no adhesive 26. Accordingly, a user can simultaneously hold the pull tab 27 of the base 2.8 and the small outer edge area of the peelable cover 20 opposite to the pull tab 27 to peel and separate the peelable cover 20 from the base 28. In this manner, the user can visually see the instruction guide 24 and access the infusion set 10.

FIGS. 4-6 illustrate the peelable cover 20 being gradually peeled from the base 28. As the peelable cover 20 is peeled, the user can see the infusion set 10 disposed in the multiple cavities 29 of the base 28. Also, the instruction guide 24 becomes more visible as the peelable cover 20 is peeled. FIG. 6 illustrates the instruction guide 24 fully visible while the peelable cover 20 is still connected to the base 28. That is, when peeling the peelable cover 20 from the base 28 to remove the infusion set 10, the peelable cover 20 does not have to be completely separated from the base 28. In an alternate embodiment, when peeling the peelable cover 20 from the base 28 to remove the infusion set 10, the peelable cover 20 is completely separated from the base 28. in either case, the peelable nature of the peelable cover 20 allows it (and the instruction guide 24) to remain intact as it is peeled from the base 28.

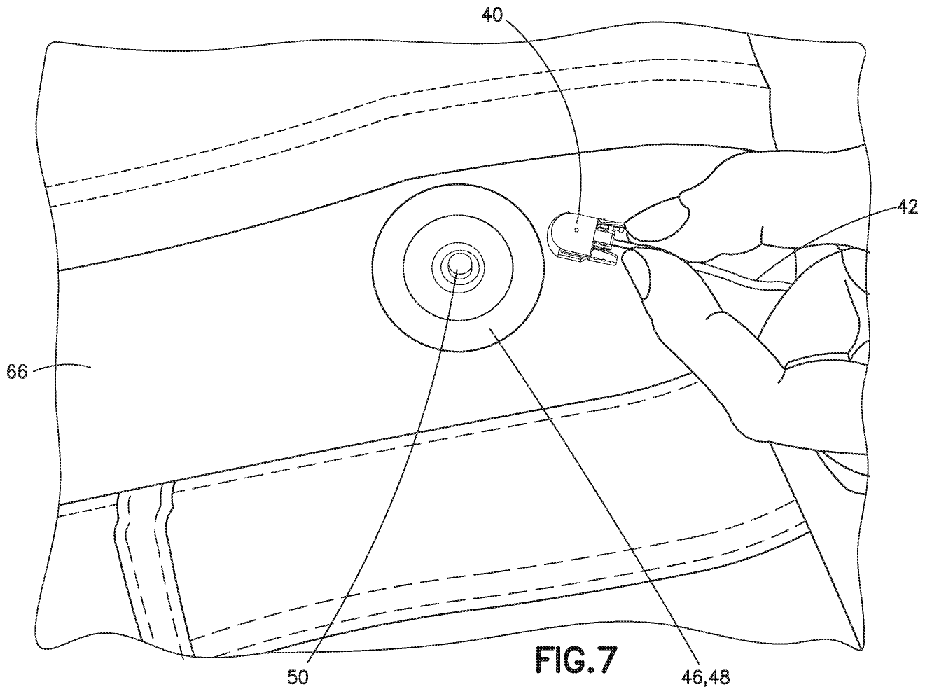

FIG. 7 illustrates the infusion set 10 being attached and secured to the skin surface 66 of the patient. Specifically, the adhesive 4$ on the adhesive patch 46 adheres to the skin surface 66. The fluid connector 40 attached to the tubing 42 is being connected to the infusion set hub 50 of the infusion set 10 to provide insulin administration.

FIG. 8 illustrates a pump 64 that is attached to the tubing 42 to supply and monitor the insulin administration to the patient. Also, FIG. 8 illustrates an inserter 62 that can be used to insert and secure the cannula 36 of the infusion set 10 into the skin surface 66 of the patient. Specifically, the infusion set 10 is placed into the inserter 62. When the distal end of the inserter 62 is pressed against the skin surface 66 of the patient, one or more buttons on the inserter 62 is depressed to activate the inserter 62. Upon activation, spring force moves the infusion set 10 toward the skin surface 66 and allows the needle and cannula 36 to pierce the skin surface 66. Thereafter, the inserter 62 and needle are removed, leaving the infusion set 10 secured to the skin surface 66 and the infusion set 10 ready for operation as illustrated in FIG. 7. After the instruction guide 24 is used, the peelable cover 20 and the base 28 are discarded or recycled.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. Any of the embodiments and/or elements disclosed herein may be combined with one another to form various additional embodiments not specifically disclosed, as long as they do not contradict each other. Accordingly, additional embodiments are possible and are intended to be encompassed within this specification and the scope of the invention. The specification describes specific examples to accomplish a more general goal that may be accomplished in another way.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges outside of the given value, for example, general tolerances associated with manufacturing, assembly, and use of the described embodiments.

1. A packaging assembly capable of carrying a medical device, the packaging assembly comprising:
a base having a cavity, the cavity being configured to carry the medical device;
a peelable cover having an inner surface and an instruction guide disposed on the inner surface; and
an adhesive disposed between the base and the inner surface of the peelable cover surrounding the instruction guide;
wherein the peelable cover is sealed to the base via the adhesive prior to use of the medical device.

2. The packaging assembly of claim I, wherein the medical device includes an infusion set having at least one of an insertion set, tubing, an adhesive patch, an adhesive, a fluid connector and a disconnect cover,

3. The packaging assembly of claim 2, wherein the insertion set includes at least one of a needle housing, a needle guard and a cannula.

4. The packaging assembly of claim 1, wherein the instruction guide comprises at least one of a printed instruction or a set of printed instructions, a written instruction or a set of written instructions, an image, a barcode, and a QR code.

5. The packaging assembly of claim 1, wherein the instruction guide includes information comprising at least one of a product specification, assembly instructions, installation instructions and operating instructions. h. The packaging assembly of claim I, wherein the instruction guide is integrated into the inner surface of the peelable cover.

7. The packaging assembly of claim 1, wherein the instruction guide is attached to the inner surface of the peelable cover.

8. The packaging assembly of claim 1, wherein the peelable cover has a fiat surface.

9. The packaging assembly of claim 1, wherein the base is clear and transparent.

10. The packaging assembly of claim 1, wherein the base is tinted, translucent or opaque.

11. The packaging assembly of claim 1, wherein the medical device includes an infusion set.

12. A method of packaging a medical device, the method comprising:
forming a base having a cavity;
placing the medical device within the cavity of the base;
applying an instruction guide to an inner surface of a peelable cover;
applying adhesive between the base and the inner surface of the peelable cover surrounding the instruction guide; and
sealing the peelable cover to the base via the adhesive.

13. The method of claim 12, wherein the medical device includes an infusion set having at least one of an insertion set, tubing, an adhesive patch, an adhesive, a fluid connector and a disconnect cover.

14. The method of claim 13, wherein the insertion set includes at least one of a needle housing, a needle guard and a cannula.

15. The method of claim 12, wherein the instruction guide comprises at least one of a printed instruction or a set of printed instructions, a written instruction or a set of written instructions, an image, a barcode, and a QR code.

16. The method of claim 12, wherein the instruction guide includes information comprising at least one of a product specification, assembly instructions, installation instructions and operating instructions.

17. The method of claim 12, wherein the instruction guide is integrated into the inner surface of the peelable cover.

18. The method of claim 12, wherein the instruction guide is attached to the inner surface of the peelable cover.

19. The method of claim 12, wherein the peelable cover has a flat surface.

20. The method of claim 12, wherein the base is clear and transparent.

21. The method of claim 12, wherein the base is tinted, translucent or opaque,

22. The method of claim 12, wherein the medical device includes an infusion set.

23. A method of using a medical device with an instruction guide, the method comprising:
peeling a peelable cover adhered to a base which encloses the medical device;
removing the medical device from the base;
viewing the instruction guide on an inner surface of the peelable cover after the peeling; and
using the medical device based on information in the instruction guide.

24. The method of claim 23, wherein the medical device includes an infusion set.

25. The method of claim 23, wherein the instruction guide is not separate from the peelable cover.

26. The method of claim 23, wherein when peeling the peelable cover from the base to remove the medical device from the base, the peelable cover is not completely separated from the base.

27. The method of claim 23, wherein when peeling the peelable cover from the base to remove the medical device, the peelable cover is completely separated from the base.

28. The method of claim 23, further comprising:
securing the infusion set to the skin of a patient.

29. The method of claim 23, further comprising:
disposing the insertion set in an inserter;
activating the inserter; and
securing the infusion set to a skin of a patient via the inserter.

30. The method of claim 23, further comprising:
discarding the peelable cover and the base after the instruction guide is used.
